(19) United States
(12) Patent Application Publication
(10) Pub. No.: US 2005/0142066 A1
(43) Pub. Date: Jun. 30, 2005

(54) Compounds or agents that inhibit and induce the formation of focal microvessel dilatations

(75) Inventor: Steven J. Mentzer, Boston, MA (US)

Correspondence Address: PALMER & DODGE, LLP;KATHLEEN M. WILLIAMS, 111 HUNTINGTON AVENUE, BOSTON, MA, 02199, US

(73) Assignee: Dana Farber Cancer Institute, Inc

(21) Appl. No.: 11/002,820

(22) Filed: Dec. 2, 2004

Related U.S. Application Data
(63) Continuation of application No. PCT/US03/17790, filed on Jun. 6, 2003.
(60) Provisional application No. 60/386,831, filed on Jun. 6, 2002.

(51) Int. Cl. A61K 49/00

(52) U.S. Cl. 424/9.2; 424/9.6

(57) ABSTRACT

The invention relates to the discovery that inflammation involves a structural modification to the microvascular architecture that results in the formation of focal microvessel dilatations or “microangiectasias.” The focal microvessel dilatations cause localized slowing of blood cell flow velocity that promotes the accumulation of extravascular lymphocytes. Methods are disclosed herein that exploit this discovery to screen for compounds that modulate the accumulation of extravascular lymphocytes. In addition, methods for treating patients suffering from cancer and diseases or pathologies associated with lymphocytic infiltration are disclosed.

### RELATED APPLICATIONS

This application is a Continuation of PCT/US03/17790, filed Jun. 6, 2003, which claimed priority to U.S. Provisional Application No. 60/386,831, filed Jun. 6, 2002; the entirety of each of which is incorporated herein by reference.

### GOVERNMENT GRANTS

At least part of the work contained in this application was performed under NIH grant HL47078. The government may have certain rights in this invention.

### FIELD OF THE INVENTION

This invention relates to the modulation of inflammation.

### BACKGROUND OF THE INVENTION

When recirculating lymphocytes migrate from the microcirculation to the extravascular site of inflammation, they must overcome the mechanical forces produced by blood flow. Blood flowing across the vascular endothelium creates shear forces at the endothelial boundary that are dependent on both flow velocity and vessel geometry. These shear forces disrupt the lymphocyte-endothelial cell adhesions necessary for transmigration. For more than a decade, the prevailing hypothesis has been that the disruptive hemodynamic forces are overcome by a multistep sequence of adhesive interactions between lymphocytes and endothelial cells (D. N. Granger, et al. (1994), J. Leukocyte Biol. 55, 662-675; T. A. Springer, et al. (1995), Annu. Rev. Physiol. 57, 827-872; S. J. Mentzer, et al. (1987), J. Cell Physiol. 130, 410-415).

Recent evidence, however, suggests that cell adhesion molecules alone cannot explain the extravascular recruitment of inflammatory lymphocytes. In normal microvessels, wall shear stresses have been estimated to be on the order of 20 dyn/cm² (H. H. Lipowsky, et al. (1978), Circ. Res. 43, 738-749; A. R. Pries, et al. (1998), Am J Physiol 275, H349-60). In simplified flow conditions, however, in vitro studies of cell adhesion indicate that lymphocyte-endothelial cell adhesions are infrequent at wall shear stresses greater than 1-2 dyn/cm² (X. Li et al. (2001), In vitro Cell Dev. Biol. 37, 599-605; X. Li, et al. (1996), Am. J. Respir. Cell Mol. Biol. 14, 398-406). These observations suggest a greater than 10-fold discrepancy between predicted wall shear stress in vivo and measured lymphocyte adhesivity in vitro.

Further, basic models of flow mechanics predict that this discrepancy should widen in inflammatory conditions. Lymphocytic inflammation is associated with an estimated 2-3-fold increase in blood flow at the peak of lymphocyte recruitment (C. He et al., (2001) In Press). The enhanced blood flow may function to meet the increased metabolic demands of the tissues, but there is an under-appreciated hemodynamic consequence as well. Without an overall increase in cross-sectional area, the increase in blood flow should substantially increase wall shear stress and decrease transendothelial migration. This is not what occurs at sites of lymphocytic inflammation. Rather, there is an increase in lymphocyte transendothelial migration. Thus, the question arises whether or not there is a structural change that occurs in the inflammatory microvasculature that aids in lymphocyte transmigration. Alternatively, is it that lymphocytes solely exhibit a multistep sequence of adhesive interactions with endothelial cells to overcome the mechanical forces produced by blood flow?

### SUMMARY OF THE INVENTION

The invention is based on the discovery that within the microcirculation an anatomical change occurs that promotes lymphocyte transmigration across the vascular endothelium. Herein, it is shown that lymphocyte slowing and transmigration in the skin are associated with focally dilated vascular segments termed “microangiectasias,” also referred to herein as “focal microvessel dilatations.” The formation of focal microvessel dilatations in the microvasculature broadens the potential for development of therapeutics to treat both cancer and inflammatory disorders. In the present invention, methods of screening for agents or compounds that inhibit or induce the formation of focal microvessel dilatations are described. In addition, methods for the treatment of cancer, as well as, methods for both the treatment and prevention of pathologies involving lymphocytic inflammation are disclosed.

Herein, focal microvessel dilatations are further shown to be associated with a proliferative and/or remodeled endothelium. The dependence of focal microvessel dilatation formation on structural adaptations of the vascular endothelium leads to an exciting alternative for the treatment of cancer: the use of pro-angiogenic factors.

Angiogenesis is the proliferation of new blood vessel growth whose progression occurs in several phases that include; the presence of an angiogenic signal, dissolution of the blood vessel basement membrane, endothelial cell proliferation, endothelial cell migration, and the formation and differentiation of capillary tubules and loops. Pro-angiogenic factors thus can be used to induce endothelial cell proliferation and potentiate focal microvessel dilatation formation thereby allowing for lymphocyte transmigration.

Pro-angiogenic factors have been used to increase vascularization of ischemic regions (U.S. Pat. No. 4,994,559). However, until now, there has been no conception for the potential use of pro-angiogenic factors in the treatment of cancer. Angiogenesis inhibitors, and not pro-angiogenic factors, have been used to inhibit neovascularization of tumors in order to inhibit both tumor growth and metastasis (Lannutti et al. (1997) Cancer Res. 57: 5277-80; O'Reilly et al. (1994) Cold Spring Harb. Symp. Quant. Biol. 59: 471-82; O'Reilly, M. S., (1997) Exs. 79: 273-94; Sim et al. (1997) Cancer Res. 57: 1329-34; Wu et al. (1997) Biochem. Biophys. Res. Commun. 236: 651-54). Thus, in contrast to prior art, which uses anti-angiogenic factors to treat tumors, the present invention discloses the use of pro-angiogenic factors for the treatment of cancer. The pro-angiogenic factors may be used to induce formation of focal microvessel dilatations in order to promote lymphocyte infiltration of a tumor and subsequent tumor cell death.

The dependence of focal microvessel dilatation formation on structural adaptations of the vascular endothelium also has therapeutic implications for treatment of inflammatory disorders. Inhibitors of lymphocyte cell-cell adhesion molecules (for example, LFA-1, ICAM-1, and L-Selectin) that have shown poor success in the past for inhibiting lymphocyte transmigration can be combined with inhibitors of focal microvessel dilatation formation, such as anti-angiogenic compounds that inhibit endothelial growth. Disclosed herein are methods for treatment of lymphocytic inflammation using a combination therapy of anti-angiogenic compounds and anti-adhesion compounds.

In one aspect, the invention encompasses a screening method for an agent or compound that inhibits the formation of focal dilatations in microvessels, comprising: contacting the microcirculation with a candidate compound under conditions that permit the formation of focal dilatations; and detecting whether a focal dilatation is formed, wherein the failure to form a focal dilatation is indicative of the inhibitory activity of the candidate compound.

In one embodiment, the method further comprises the step of administering the compound to an animal and determining whether focal microvessel dilatation formation is inhibited at a site of inflammation. In another embodiment, the inhibition of focal microvessel dilatation reduces the accumulation of perivascular lymphocytes at the site of inflammation. In another embodiment, the method further comprises administering an inhibitor of lymphocyte cell-cell adhesion.

In another aspect, the invention encompasses a screening method for an agent or compound that inhibits the formation of focal dilatations in microvessels, comprising: contacting the microcirculation with a candidate compound under conditions that permit the formation of focal dilatations in microvessels; and detecting one or more of the following: a) substantially no focal reduction in blood cell flow velocity within the microvasculature; b) substantially no reduction in wall shear stress within the microvasculature; c) substantially no focal increase in the diameter of the microvasculature; d) substantially no increase in extravascular lymphocytes; and e) substantially no increase in endothelial cell proliferation, wherein the detection of at least one of (a), (b), (c), (d) or (e), relative to microcirculation not contacted with the candidate compound, indicates that the candidate compound inhibits the formation of focal dilatations in microvessels.

In one embodiment, the flow velocity is detected for a blood cell which is one or more of a lymphocyte, neutrophil or red blood cell. In another embodiment, the lymphocyte, neutrophil or red blood cell is detectably labeled. In yet another embodiment, the lymphocyte, neutrophil or red blood cell is fluorescently labeled.

In another aspect, the invention encompasses a screening method for an agent or compound that induces the formation of focal dilatations in microvessels, comprising: contacting the microcirculation with a candidate compound under conditions that permit the formation of focal dilatations in microvessels; and detecting whether a focal dilatation in a microvessel is formed, wherein the formation of a focal dilatation in a microvessel is indicative of the induction activity of the compound.

In one embodiment, the method further comprises the step of administering the compound to an animal with a tumor and detecting the formation of focal microvessel dilatations adjacent to the tumor. In another embodiment, the induction of the focal microvessel dilatations increases perivascular lymphocyte accumulation within the tumor.

In another aspect, the invention encompasses a screening method for an agent or compound that induces the formation of focal dilatations in microvessels, comprising: contacting the microcirculation with a candidate compound under conditions that permit the formation of focal dilatations in microvessels; and detecting one or more of the following: a) a reduction in blood cell flow velocity within the microvasculature; b) a reduction in wall shear stress within the microvasculature; c) a focal increase in the diameter of the microvasculature; d) an increase in extravascular lymphocytes; and e) an increase in endothelial cell proliferation, wherein the detection of one or more of (a), (b), (c), (d) or (e), relative to microcirculation not contacted with the candidate compound, indicates that the candidate compound induces the formation of focal dilatations in microvessels.

In one embodiment, the flow velocity is detected for a blood cell which is one or more of a lymphocyte, neutrophil or red blood cell. In another embodiment, the lymphocyte, neutrophil or red blood cell is detectably labeled. In yet another embodiment, the lymphocyte, neutrophil or red blood cell is fluorescently labeled.

In another aspect, the invention encompasses a method of screening for a compound that modulates the accumulation of extravascular lymphocytes, the method comprising: a) contacting a tissue with an agent that induces inflammation; b) contacting the tissue with a candidate modulator compound; and c) detecting a difference in one or more of the following: i) local accumulation of extravascular lymphocytes in the tissue; ii) the size or number of focal microvessel dilatations in microvessels in the tissue; iii) localized blood cell flow velocity in a microvessel of the tissue; iv) wall shear stress within the microvasculature; and v) endothelial cell proliferation, relative to the detection of one or more of (i), (ii), (iii), (iv) and (v) occurring in the absence of the candidate compound, wherein the difference indicates that the candidate compound modulates the accumulation of extravascular lymphocytes.

In one embodiment, the difference comprises a decrease in one or more of (i), (ii) and (v), and/or an increase in one or both of (iii) and (iv), and the difference indicates that the candidate modulator is an inhibitor of the accumulation of extracellular lymphocytes.

In another embodiment, the difference comprises an increase in one or more of (i), (ii) and (v), and/or a decrease in one or both of (iii) and (iv), and the difference indicates that the candidate modulator is an inducer of the accumulation of extracellular lymphocytes.

In another embodiment, the difference in blood cell flow velocity is detected for a blood cell which is one or more of a lymphocyte, neutrophil or red blood cell. In another embodiment, the difference in blood cell flow velocity is measured using intravital video microscopy. In another embodiment, the lymphocyte, neutrophil or red blood cell is detectably labeled. In yet another embodiment, the lymphocyte, neutrophil or red blood cell is fluorescently labeled.

In one aspect, the detection of focal microvessel dilatations can be used to validate a compound identified in a high throughput screen as an in vivo modulator of extravascular lymphocyte accumulation. In one aspect, microarray hybridization analyses using cDNAs from tissue with and without inflammation (e.g., lymphocytic inflammation) can be used to identify genes differentially expressed during inflammation. Such microarray hybridization analyses are well known to those skilled in the art. The products of such differentially regulated genes can then be used in high throughput screens of various compounds (peptides, polypeptides, small molecules, etc.) to identify compounds that bind the differentially regulated gene product(s). Again, high throughput screens for compounds that bind a given gene product are well known to those skilled in the art. A compound identified in this or another manner as a potential modulator of inflammation can then be validated as an in vivo modulator of such inflammation by contacting the compound with a tissue, either alone, or with an agent that induces inflammation, and detecting focal microvessel dilatations, a focal difference in microvessel blood cell flow velocity, a difference in extravascular lymphocyte accumulation, a difference in wall shear stress, or a difference in endothelial cell proliferation relative to tissue not treated with the compound. An increase in one or more of focal microvessel dilatations, extravascular lymphocyte accumulation, or endothelial cell proliferation, or a decrease in one or both of focal microvessel blood cell flow velocity or wall shear stress is indicative that the compound increases lymphocytic transmigration. A decrease in one or more of focal microvessel dilatations, extravascular lymphocyte accumulation, or endothelial cell proliferation, or an increase in one or both of focal microvessel blood cell flow velocity or wall shear stress is indicative that the compound decreases inflammation.

In accord with this aspect, the invention further encompasses a method of validating a candidate compound as an anti-inflammatory compound, the method comprising: a) contacting a tissue with an agent that induces inflammation; b) contacting the tissue with a compound identified as a candidate anti-inflammatory compound in a high throughput assay; c) detecting a difference in local accumulation of extravascular lymphocytes in the tissue relative to the local extravascular lymphocytic accumulation occurring in the absence of the candidate compound, wherein the difference validates the candidate compound as an anti-inflammatory compound.

In one embodiment, the high throughput assay comprises microarray hybridization.

In another aspect, the invention encompasses a method for treating a tumor in a patient by administering a pro-angiogenic factor in an amount sufficient to induce the formation of focal microvessel dilatations and lymphocytic infiltration of the tumor.

In another aspect, the invention encompasses a method of treating a patient suffering from a disease involving inflammation, e.g., lymphocytic inflammation, comprising administering an inhibitor of angiogenesis and an inhibitor of lymphocyte cell-cell adhesion in an amount sufficient to inhibit the formation of focal microvessel dilatations and lymphocytic infiltration into the site of inflammation.

In one embodiment, the inhibitor of lymphocyte cell-cell adhesion is selected from the group consisting of: an inhibitor of one of LFA-1, I-CAM 1, and L-selectin.

In another embodiment, the patient suffers from a disease selected from the group consisting of: psoriasis, eczema, atopic dermatitis, pityriasis rosea, mycosis, fungoides, lichen planus, and granuloma annulare. In another embodiment, the patient has an autoimmune disease.

In another aspect, the invention encompasses a method for immunosuppression in a patient comprising administering an angiogenesis inhibitor and an inhibitor of lymphocyte cell-cell adhesion in an amount sufficient to inhibit the formation of focal microvessel dilatations and lymphocytic infiltration of a site of inflammation.

In one embodiment, the inhibitor of lymphocyte cell-cell adhesion is selected from the group consisting of: an inhibitor of one of LFA-1, I-CAM 1, and L-selectin.

In another embodiment, the patient has a transplanted organ.

### BRIEF DESCRIPTION OF THE FIGURES

The objects and features of the invention can be better understood with reference to the following detailed description and accompanying drawings.

FIG. 1 shows intravital fluorescence videomicroscopy of the dermal microcirculation 96 hours after oxazolone stimulation. A) A representative dark field video microscopy image of the sheep skin 96 hours after oxazolone stimulation. The circles identify focal regions associated with lymphocyte slowing and transmigration. B) A single videomicroscopy image showing five fluorescently labeled lymph-derived cells passing through a focal region of the microcirculation. C) Digital quasi-3D recombination of videomicrographs showing 30 cells passing through the focal region associated with slowing and transmigration (bar=60 μm). The volumetric 3D reconstruction of 180 images (30 fps) provides an outline of an apparent focal microvessel dilatation. The arrow shows the direction of blood flow and the image of a transmigrated cell.

FIG. 2 shows velocities of lymph-derived cells passing through a focal area of the oxazolone-stimulated dermal microcirculation. Instantaneous cell velocities were calculated at 33 msec intervals over the axial distance arbitrarily extending 50 um on either side of the midpoint of the region.

FIG. 3 shows a velocity-location map of lymphocytes passing through a representative focal microvessel dilatation after a single carotid artery injection of labeled lymphocytes. The overall flow direction is from left to right. Velocities were calculated at 33-msec intervals, shading-coded as indicated, and plotted on a 200 um×200 um grid. Focal microvessel dilatations were functionally identified by lymphocyte velocities <0.68 um/sec.

FIG. 4 shows the velocities of fluorescently labeled lymphocytes traversing focal microvessel dilatations 96 hours after the application of oxazalone, based on observations of 600 cells in four sheep. Instantaneous cell velocities were measured 150 um upstream of the functional midpoint of the focal microvessel dilatation (afferent), at the midpoint (midpoint), and 150 um beyond (efferent). Boxes represent 25-75% significant (p<0.0001 by Student's t test). Because of spatial limitations of the optical fields and temporal limitations of the video streaming, cell velocities above 5 um/msec could not be reliably measured; therefore, the indicated afferent and efferent velocity ranges may be underestimates.

FIG. 5 is a scanning electron microscopy image of the inflammatory microcirculation. Scanning electron micrographs are shown of the control (A,C) and oxazolone-stimulated (B,D) microcirculation 96 hours after the application of the oxazolone. The microcirculatory topology was analyzed using stereo-pair scanning electron microscopy (SEM) of microvascular corrosion casts. Representative examples of low (A,B; bar=200 um) and high (C,D; bar=50 um) magnification SEMs are shown.

FIG. 6 shows microhemodynamic mapping of wall shear stress in a representative focal microvessel dilatation and estimation of wall shear stress variations in such microvessel dilatations. A. Gradual transition in diameter. B. Abrupt transition. In each case, the assumed vessel shapes are given by rotating the shaded region about the axis of symmetry (dash-dotted line). Arrows indicate approximate streamlines of flow. The graphs show the corresponding variation of fluid shear stress on the outer wall. In each case, wall shear stress is 15.5 dyn/cm2 in the afferent segment and approaches 1.08 dyn/cm2 in the dilated segment. The locations of minimum wall shear stress are indicated.

FIG. 7 is a temporal map of a focal microvessel dilatation. An 8 second recording interval was used to track lymphocytes through a microangiectasia, 23 lymphocytes passed through this region of the microcirculation. The 240 images obtained during this recording interval were assigned a sequential gray scale level (progressing from black to white). The multiple images were then digitally recombined to provide a “temporal area map” of the blood vessel (which can be readily pseudocolored for analysis) (Panel A). A derived “outline” of the vascular segment is shown in Panel B).

FIG. 8 shows that anti-angiogenic factors inhibit lymphocyte transmigration. The number of lymphocytes recruited per 200×400 um grid in the inflammatory and steroid-treated skin are shown.

### DETAILED DESCRIPTION

The invention is based upon the observation of a structural change that occurs in the microcirculation that may be inhibited or induced in order to alter lymphocyte transmigration across blood vessels. The structures are known as focal microvessel dilatations or “microangiectasias” and they represent regions of focal dilation, lymphocyte slowing, reduced wall shear stress, lymphocyte transmigration, and endothelial cell proliferation and/or hypertrophy, all of which can be measured.

In order to more clearly and concisely describe and point out the subject matter of the claimed invention, the following definitions are provided for specific terms which are used in the following written description and the appended claims.

Definitions

“Focal microvessel dilatations” (“microangiectasias”) are distinct anatomical structures that herein have been found to occur within the microcirculation of mammals. The formation of a “focal microvessel dilatation” is characterized by a focal dilation, or ballooning, of the microvessels. The presence of an anatomical change in the vasculature was first hypothesized upon an observation that there are distinct focal regions within the microcirculation that are associated with the slowing and transmigration of lymphocytes. When lymphocytes migrate from the microcirculation to an extravascular site of inflammation, they need to overcome the mechanical forces of blood flow, for example, the shear force created by blood flowing across the vascular endothelium. It is the shear forces produced by blood flow that disrupt the lymphocyte-endothelial cell adhesions necessary for transmigration of lymphocytes across the vessel endothelium. The prevailing dogma has been that lymphocytes overcome this high shear force solely by exhibiting a multi-step adhesive interaction with endothelial cells. However, additionally, an anatomical change, or dilation, of microvessels occurs that locally reduces the blood flow velocity and the resultant shear force aiding in lymphocyte transmigration.

As used herein, “focal microvessel dilatations” (“microangiectasias”) are areas of focal venular dilation, readily recognized by one of skill in the art, that are found within the inflammatory microvasculature. The presence or formation of a “focal microvessel dilatation” is defined by one or more of i) a localized increase of at least two-fold in microvessel diameter across any cross-sectional orientation of the vessel—on either side of a “focal microvessel dilatation” the diameter of the microvessel is in the normal range of 10-20 um, ii) the presence of perivascular or extravascular lymphocytes or lymphocyte transmigration, iii) a localized decrease in blood cell flow velocity (which is necessarily accompanied by a decrease in wall shear stress), and iv) a proliferative/hypertrophic vascular endothelium.

A “focal microvessel dilatation” can range up to about 90 um in diameter. Histologic studies show that “focal microvessel dilatations” are associated with a proliferative endothelium.

The “focal microvessel dilatations” tend to be located at about 100 uM intervals apart from each other in regions of inflammation, for example, in skin tissue. Morphological studies demonstrate that the area of vessel dilation has frequently greater than a 2-fold increase in lumenal diameter, for example, 3-fold or more. The increase in the lumenal diameter of focal microvessel dilatations locally reduces the wall shear stress to below 3 dyn/cm². Normal, non-dilated microvessels have a wall shear stress on the order of 15-20 dyn/cm².

Without being bound to any one mechanism, it is believed that the localized reduction in blood cell flow velocity and the resulting localized reduction in wall shear stress of a focal microvessel dilatation facilitates lymphocyte transmigration across the endothelium to an extravascular site of inflammation.

The formation of focal microvessel dilatations within the inflammatory microvasculature has many implications for the therapeutic treatment of patients suffering from diseases involving inflammation, e.g., lymphocytic inflammation. Compounds or agents that inhibit focal microvessel dilatation formation can be used to inhibit lymphocyte transmigration at the site of lymphocytic inflammation or to prevent acute rejection, for example, after organ transplantation. Furthermore, agents or compounds that induce focal microvessel dilatation formation can be used in the treatment of cancer by inducing lymphocytic infiltration of tumors. Thus, compounds that affect focal microvessel dilatation formation can be used for treatment of both inflammatory diseases and cancer. The present invention describes methods for screening of compounds or agents that inhibit or induce focal microvessel dilatation formation.

As used herein, “formation of a focal microvessel dilatation” (or “formation of a microangiectasia”) can be defined by the presence of at least one of the definitional characteristics of a focal microvessel dilatation. The “formation of a focal microvessel dilatation” is “detected” by the observation of at least one of the definitional characteristics.

As used herein, the term “acute” means that the diameter of the vessel changes abruptly, rather than gradually. An abrupt change is a change wherein the diameter of the vessel at least doubles over a length of the vessel no greater than the original or minimal diameter of the vessel before the change in diameter.

As used herein, the term “inflammation” refers to the presence of tissue damage in an individual. For example, the tissue damage can result from autoimmune processes, microbial infection, tissue or organ allograft rejection, neoplasia, idiopathic diseases or such injurious external influences as heat, cold, radiant energy, electrical or chemical stimuli, or mechanical trauma. Regardless of the cause, the inflammatory response generally comprises an intricate set of functional and cellular changes, involving modifications to microcirculation (including focal microvessel dilatation formation), accumulation of fluids, and the influx and activation of inflammatory cells (e.g. lymphocytes).

As described herein, an “increase in diameter” of a microvessel represents at least a 2 fold increase in diameter in any cross sectional dimension as compared to the normal microvessel diameter range of 10-20 um.

As described herein, a “reduction in blood cell flow velocity” refers to at least a 10-fold reduction in velocity as compared to that observed in undilated microvessels, which is 3 um/msec or greater. A reduction in blood cell flow velocity includes a localized reversal of blood flow, i.e., the occurrence of a “back eddy” in within the ballooned portion of a focal microvessel dilatation. A “localized” reduction in blood cell flow velocity refers to a reduction in blood cell flow velocity in a limited area of the vessel, such that the blood cell flow velocity on either side of the limited area is generally 2-3 um/msec or greater. In skin, the distinguishing characteristic of the focal microvessel dilatations is the acute change in vessel diameter: an abrupt increase in vessel diameter is associated with slower flow and decreased wall shear stress. In contrast, a gradual change in microvessel diameter does not have this effect.

As described herein, a “decrease in wall shear stress” is indicative of focal microvessel dilatation formation. Herein, a “decrease” is considered greater than a 5-fold decrease in wall shear stress as compared to the wall shear stress of normal microvessels, which ranges from 20 to 100 dyn/cm².

As described herein, “extravascular lymphocyte accumulation” refers to the presence of regional lymphocytic perivascular clusters of lymphocytes, which is indicative of the presence of a focal microvessel dilatation. The presence of lymphocytic perivascular clusters may be measured by injecting labeled lymphocytes into the microcirculation at discrete time points. A “difference” in the accumulation of extravascular lymphocytes is an increase or decrease in extravascular lymphocyte accumulation. An “increase in extravascular lymphocyte accumulation” means at least a 2 fold increase, preferably at least a 3-, 5-, 10-fold or greater increase in the number of extravascular lymphocytes detected in a tissue region exposed to a test compound relative to a region not exposed to that compound. A “decrease in extravascular lymphocyte accumulation” means at least a 2-fold decrease, and preferably at least a 3-, 5-, 10-fold or greater decrease in the number of extravascular lymphocytes in a tissue region contacted with a test compound and an inducer of inflammation, relative to a tissue region contacted with the inducer of inflammation alone.

As used herein, an “increase in lymphocyte transmigration” refers to at least a 10-fold increase in the transmigration frequency of lymphocytes across the endothelium in comparison to basal level rates which can range from 10²-10³ lymphocytes per minute. As used herein, endothelial cell “proliferation” can refer to endothelial cell division or to a change in size of the endothelial cell. Endothelial cell proliferation can be monitored using cell cycle-specific markers.

As used herein, a “failure to form a focal microvessel dilatation” can be defined by the absence of at least one of the definitional characteristics of a focal microvessel dilatation. Herein, “inhibition” of microangiectasia formation refers to inhibiting microangiectasia formation such that the wall shear stress of the microvasculature remains above 3 dyn/cm².

As used herein, a “difference in the number or size” of focal microvessel dilatations refers to an increase or decrease in the number or size of microvessel dilatations in a tissue contacted with a candidate modulator relative to a tissue not contacted with that candidate modulator. An increase or decrease in number is by at least 50%. Similarly, an increase or decrease in size is at least a 50% increase or decrease in focal dilatation diameter.

As used herein, “reducing the amount of lymphocytic infiltration” refers to preventing lymphocytic transmigration across the microvasculature endothelium such that the rate of transendothelial migration is less than the rate observed in acute rejection which is on the order of more than 10⁶ lymphocytes per minute. Further, “reducing” the amount of lymphocytic infiltration refers to preventing lymphocytic transmigration across the microvasculature endothelium such that lymphocytic inflammation is subdued.

As used herein, “microcirculation” refers to the vascular network lying between the arterioles and venules. The “microcirculation” includes capillaries, metarterioles and arteriovenous anastomoses, venules, and the flow of blood through this network. The “inflammatory microcirculation” refers to areas of the microcirculation where lymphocytes can transmigrate.

As used herein, “microvasculature” or “microvessels” refer to venules, capillaries, metarterioles and arteriovenous anastomoses.

As used herein, the modifier “substantially no” when applied to an increase, reduction or decrease means that there is less than a 5% change in the value being measured relative to a reference, e.g., less than a 5% change in the value being measured in a tissue treated with a compound, relative to that value detected in a tissue not treated with the compound.

As used herein, “patient” is identified as human or animal.

As used herein, “organ transplant rejection” is defined with reference to lymphocyte mediated immune response. In “organ transplant rejection” there is an increase in blood flow to a transplanted organ. The increase in blood flow is associated with increased tissue edema and impaired organ function. In addition, “organ transplant rejection” results in accumulation of lymphocytes in the perivascular tissues. The rate of transendothelial migration of lymphocytes can be greater than 10⁶ lymphocytes per minute upon acute “organ transplant rejection”. “Organ transplant rejection” may be tested by biopsy and assessment of the presence or absence of perivascular lymphocytic infiltration around vessels.

As used herein, “immunosuppression” refers to prevention of a lymphocyte mediated immune response. As used herein, lymphocytes refer to B or T-cells, wherein, T-cells may be helper T-cells or cytotoxic T-cells.

Herein, representative diseases of “lymphocytic inflammation” include, but are not limited to autoimmune diseases such as articular rheumatism, systemic lupus erythematosus, Sjoegren syndrome, multiple sclerosis, myasthenia gravis, type I diabetes mellitus, endocrine ophthalmic disease, primary biliary cirrhosis, Crohns disease, glomerular nephritis, sarcoidosis, psoriasis, eczema, atopic dermatitis, pityriasis rosea, mycosis, fungoides, lichen planus, and granuloma annulare, variola, hypoplastic anemia, idiopathic thrombocytopenic purpura, rheumatoid arthritis, and the like. Herein, “lymphocytic inflammation” can occur in graft vs host disease and in viral diseases, such as Herpes Simplex Virus, Varicella, and Herpes Zoster.

Herein, representative examples of pro-angiogenic factors include, but are not limited to, vascular endothelial derived growth factor (VEGF, GenBank Accession No. NM003376), angiogenin (GenBank Accession No. M11567), angiopoietin-1 (GenBank Accession No. AY124380), Del-1 (GenBank Accession No. U70312), fibroblast growth factors: acidic (aFGF; GenBank Accession No. E03043) and basic (bFGF; GenBank Accession No. E05628), follistatin (GenBank Accession No. BC004107), granulocyte colony-stimulating factor (G-CSF; GenBank Accession No. E01631), hepatocyte growth factor, (HGF)/scatter factor (SF; GenBank Accession No. AY246560), Interleukin-8 (IL-8; GenBank Accession No. NM000584), leptin (GenBank Accession No. NM000230), midkine (GenBank Accession No. NM002391), placental growth factor (GenBank Accession No. NM002632), platelet-derived endothelial cell growth factor (PD-ECGF; GenBank Accession No. NM001953), platelet-derived growth factor-BB (PDGF-BB; GenBank Accession No. X63966), pleiotrophin (PTN; GenBank Accession No. NM00285), proliferin (GenBank Accession No. XM193114), transforming growth factor-alpha (TGF-alpha; GenBank Accession No. BT006833), transforming growth factor-beta (TGF-beta; GenBank Accession No. BT007245), tumor necrosis factor-alpha (TNF-alpha; GenBank Accession No. M16441), and the like. Many of these factors are commercially available from various sources. Pro-angiogenic factors may also be small molecules or proteins, not normally present in the body.

Herein, representative examples of anti-angiogenic factors include, but are not limited to, thaloidomide, steroids, angiostatin (plasminogen fragment, GenBank Accession No. P20918 (amino acid sequence), antiangiogenic antithrombin III (GenBank Accession No. AH004913), cartilage-derived inhibitor (CDI; Moses & Langer, 1991, J. Cell. Biochem. 47: 230-5 (1991)), CD59 complement fragment (GenBank Accession No. BT007104), endostatin (collagen XVIII fragment; GenBank Accession No. NM130445), fibronectin fragment (GenBank Accession No. BT006856), gro-beta (GenBank Accession No. M36820), heparinases (GenBank Accession No. NM006665), heparin hexasaccharide fragment, human chorionic gonadotropin (hCG; GenBank Accession No. V00518), interferon alpha (GenBank Accession No. NM024013)/beta (GenBank Accession No. NM002176)/gamma (GenBank Accession No. AY255837), interferon inducible protein (IP-10), interleukin-12 (GenBank Accession No. NM000882), kringle 5 (plasminogen fragment; GenBank Accession No. NM000301), metalloproteinase inhibitors (TIMPs; e.g., GenBank Accession Nos. NM000362, NM003254, NM003255), 2-Methoxyestradiol, placental ribonuclease inhibitor, plasminogen activator inhibitor (GenBank Accession No. NM006216), platelet factor-4 (PF4; (GenBank Accession No. NM002619), prolactin 16 kD fragment (GenBank Accession No. NM000948), proliferin-related protein (PRP; GenBank Accession No. NM053364), retinoids, tetrahydrocortisol-S, thrombospondin-1 (TSP-1, GenBank Accession No. NM003246), transforming growth factor-beta (TGF-β; GenBank Accession No. BT007245), vasculostatin, vasostatin (calreticulin fragment; GenBank Accession No. AY047586), and the like. A number of these factors are available commercially.

Anti-angiogenic factors may also be small molecules and obtained from natural sources, including: tree bark, fungi, shark muscle and cartilage, sea coral, green tea, and herbs (licorice, ginseng, cumin, garlic).

Herein, representative inhibitors of lymphocyte cell-cell adhesion include, but are not limited to, “inhibitors” of ICAM-1, LFA-1, and L-selectin. The “inhibitor” may be, for example, a small molecule, antibody, DNA, RNA, or protein. Herein “inhibitor” means any molecule that can either induce an inhibitor or directly inhibit the normal function of cell-cell adhesion molecules, for example, ICAM-1, LFA-1, and L-selectin. Herein, an “inhibitor of lymphocyte cell-cell adhesion” can be any molecule that directly binds an adhesion receptor, that inhibits expression of an adhesion receptor, or that inhibits activation of cell adhesion ligands.

Many anti-LFA-1, anti-CAM, anti-VLA-4, and anti-selectin antibodies have been described in the literature are useful in the present invention (Yusuf-Makagiansar et al. (2000) Curr Top Biochem Res., 2: 33-49; Gonzalez-Amaro et al (1998) J. Immunol., 161: 65-72; Cavazzana-Calvo et al (1995) Transplantation, 59: 1576-1582; Hourmant et al (1996) Transplantation, 62: 1565-1570; Isobe et al (1994) J. Immunol., 153: 5810-5818; Samacki et al (2000) Gut, 47: 97-104; Lobb et al (1996) Acad. Sci., 796: 113-123; Yednock et al (1992) Nature, 356: 63-66; Molina et al (1994) J. Immunol., 153: 2313-2320).

Example peptide and small molecule cell-cell adhesion inhibitors include, but are not limited to, cyclic ICAM-1-derived peptides (i.e. cIBR and cLAB.L), peptides derived from functional regions of ICAM-1 (i.e. residues 367-394, Ala378) and peptides from the alpha- and beta-subunits of LFA-1. Synthetic peptides and peptide-like substances (i.e. peptidomimetics) that possess the amino acid motifs recognized by 31- and 132-integrins may also be used to block leukocyte adhesion. For example, cyclic peptides containing the LDV sequence are potent inhibitors of VLA-4 mediated adhesion.

Examples of inhibitors of cell adhesion molecule expression include, but are not limited to, salicylates, methotrexate, and pentoxifylline. In addition, suitable examples of inhibitors of cell adhesion molecule activation, include, but are not limited to, indomethacin, aceclofena, and diclofenac.

How to Determine Regional Dilation of Microvessels

The formation of a focal microvessel dilatation can be determined by the observation of an acute increase in microvessel diameter. Indications of focal microvessel dilatation formation can be obtained from microscopic illumination from a variety of sources (transillumination or epi-illumination). To identify the detailed structure of the microangiectasia focal regions, a corrosion casting technique has been developed that can perfuse the entire microcirculation (see below). This technique was necessary because of the significant arteriovenous interconnections that develop during inflammation. Scanning electron microscopy of the casts has demonstrated focal areas of venular dilatation. In the control circulation, these microvessels are typically 10-20 um in diameter. The comparable regions examined 96 hours after antigen-stimulation demonstrate balloon-like dilatation up to 50-90 um in diameter. Herein, focal microvessel dilatation formation can be monitored by the observation of an increase in a regional diameter of the microvasculature. As described herein, an increase represents, at least a 2 fold increase in diameter in any cross sectional dimension as compared to normal microvessel diameter range of 10-20 um.

The following is an exemplary method for corrosion casting. After systemic heparinization with 750 u/kg intravenous heparin, external auricular arteries are bilaterally cannulated and perfused with approximately 100 cc of 37° C. saline followed by a 2.5 percent buffered glutaraldehyde solution (Sigma) at pH 7.40. The casts can be made by perfusion of ear arteries with 100 cc of Mercox (SPI, West Chester Pa.) diluted with 20 percent methylmethacrylate monomers (Aldrich Chemical, Milwaukee Wis.). After complete polymerization, the ears are harvested and macerated in 5% potassium hydroxide followed by drying and mounting for scanning electron microscopy. The microvascular corrosion casts can be imaged after coating with gold in Argon atmosphere with a Philips ESEM XL30 scanning electron microscope.

How to Determine Blood Cell Flow Velocity

The formation of a focal microvessel dilatation can also be determined by the observation of a decrease in blood cell flow velocity within a focal region of a microvessel. The focal dilation of a microvessel has an impact on the regional microhemodynamics. The effect can be illustrated using a river analogy. A sudden widening of a river, of the relative magnitude of a focal microvessel dilatation, results in a dramatic slowing of any object in the flow stream. Lymphocyte slowing can be monitored by intravital videomicroscopy studies as described in, West et al. (2001), Am. J. Physiol. Heart Circ. 281: H1742-H1750. To optimize visualization, lymphocytes, redblood cells, neutrophils, or other particles in the size range of these cells are fluorescently labeled. The fluorescent labeling of migratory lymphocytes leaving the antigen-stimulated lymph node has allowed the tracking of their migration into the antigen-stimulated skin and lung. Using epi-fluorescence video microscopy, the movement of lymphocytes or other labeled cells or particles in the tissue can be tracked and recorded. These intravital microscopy recordings were the initial demonstration of “recruitment-associated venules.” Using these methods, it has been shown that lymphocytes move through tissues at velocities in excess of 3 um/msec. In microangiectasia focal regions, the lymphocytes dramatically slow, for example, to velocities less than 0.3 um/msec. Herein, a reduction in lymphocyte velocity is at least 10-fold as compared to that normally observed in the absence of a focal microvessel dilatation, which is 3 uM/msec or higher.

How to Determine Wall Shear Stress

Another measure of focal microvessel dilatation formation is the observation of a decrease in wall shear stress of a microvessel. The local dilation of a microvessel has an impact on the wall shear stress. The abrupt decrease in flow velocity in dilated vascular segments produce a marked decrease in shear rates. Wall shear stresses are dependent upon cell velocity and vessel geometry.

Flow patterns within the focal microvessel dilatation can be visualized using fluorescent tracers of plasma flow, red cells, lymphocytes and neutrophils. The following parameters are typically monitored when evaluating routine microcirculatory measurements; Diameter (um), Q (nl/sec), V_{RBC} (um/sec), V_{lymphocyte} (um/sec), T_{w}(dyn/cm2), V_{rolling} (um/sec), Vₘₑₐₙ (um/sec), and L_{flux} (cell/sec), wherein Q is the volumetric flow rate, V_{RBC} (um/sec) is velocity of RBC, V_{lymphocyte} (um/sec) is velocity of lymphocyte, T_{w}(dyn/cm2) is the shear stress, V_{roling} (um/sec) is a measure of marginated leukocytes, Vₘₑₐₙ (um/sec) is mean velocity, and L_{flux} (cell/sec) is a measure of lymphocyte transmigration. The microhemodynamic assessments in focal microvessel dilatations described herein are based on similar parameters, but the complex flow conditions require computer and mathematical simulations described in more detail below.

Flow patterns and wall shear stress can be assessed in vivo using flow tracers. The analysis of spatial variations in blood flow using fluorescent plasma tracer has several methodological advantages in investigating focal microvessel dilatations. First, the single injection technique has been used in vivo (Burbank et al. (1984). Journal of the American College of Cardiology 4: 308-315) and has been validated in a single input system (Nobis et al. (1985). Microvasc. Res. 29: 295-304.). Second, the injection technique permits an assessment of local plasma flow in the focal microvessel dilatations. The direct visualization of the focal microvessel dilatations permits the mapping of flow redistribution at the site of lymphocyte transmigration (West et al., Spatial variation in plasma flow after oxazolone stimulation, Inflammation Res., in press). Third, the direct measurement of emitted light obviated the need for blood sampling and eliminated the errors in downstream venous sampling. Fourth, the use of fluorescence intravital videomicroscopy offers the possibility of multi-color fluorescence labeling of lymphocyte and RBC blood elements (He et al., (2001) J. Histochem. Cytochem. 49: 511-518.). Multi-color labeling may permit the near-simultaneous correlation of lymphocyte flux and blood flow calculations.

Lymphocytes and peripheral blood red cells are collected, differentially labeled with fluorescent dyes (e.g. green=lymphocytes; red=red cells) and injected into the feeding microcirculation. The lymphocytes are “biologically relevant” as they are obtained from the efferent lymph draining the inflammatory tissue. Intravital microscopy is used to separately record the movements of lymphocytes and red cells in the microcirculation. In the lung, recordings are obtained only from the inflammatory tissue (because of the unilateral “thoracic window”). At the beginning and end of each injection period, plasma marker (FITC-dextran) is injected to define the topography of the network (He et al., (2001) Spatial variation of plasma flow in the oxazolone-stimulated microcirculation, Inflammation res., in press). The skin provides a useful control for lung intravital microscopy because comparisons are made between the inflammatory and control microcirculations during each recording period. The tissue is harvested (h) at the conclusion of the experimental period to histologically confirm the observations by intravital microscopy.

The measurement of microcirculatory spatial hemodynamics is obtained by intravital microscopy and motion analysis software algorithms. The movement of the fluorescently labeled cells is recorded as they pass through the tissue using intravital microscopy. Further hemodynamic information can be obtained from plasma marker and labeled red blood cell injections. The videomicroscopy recordings can be analyzed for blood flow and cell velocity as well as cell movements (time-location maps). Specific structural regions of a microcirculation are identified by plasma marker injections as well as temporal area maps (Li X. et al. (1996). Am. J. Respir. Cell Mol. Biol. 14: 398-406, Li X et al., (2001), Mentzer S J. In vitro Cell Dev. Biol. In press; West C. A., et al. (2001c). Am. J. Physiol. Heart Circ. 281: H1742-H1750; West C, et al. (2001) Spatial variation of plasma flow in the oxazolone-stimulated microcirculation. Submitted; He C, et al. (2001). J. Appl. Physiol. Submitted.). FIG. 7 shows an example of a temporal map; using an 8 second recording interval, 23 lymphocytes passed through this region of the microcirculation. The 240 images obtained during this recording interval were assigned a sequential gray scale level (progressing from black to white). The multiple images were then digitally recombined to provide a “temporal area map” of the blood vessel (which can be readily pseudocolored for analysis) (Panel A). A derived “outline” of the vascular segment is shown in Panel B). In one embodiment, the experimental design uses awake and spontaneously ventilating sheep to insure stable hemodynamics.

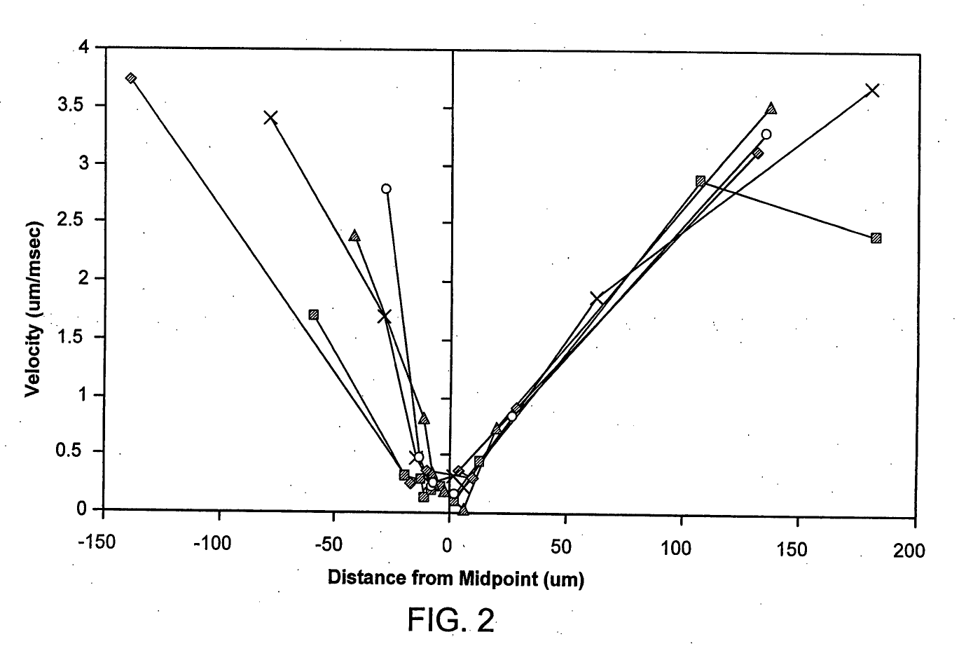

The calculation of wall shear stress is determined by finite-element computations of flow fields in the neighborhood of the transition from the afferent vessel to the dilated segment. For computation of wall shear stress a finite element program (FlexPDE, PDE Solutions Inc, Antioch Calif.) is used to solve for Stokes flow (i.e., flow of a Newtonian fluid with fixed viscosity and negligible inertia) in an axisymmetric geometry. The shape of the vessel wall in the transition region consists of parts of two ellipses, matched to give continuous slopes. The center-line flow velocity in the afferent vessel was assumed to be 2 mm/s, in the range of the lymphocyte velocities observed in this region (FIG. 2), corresponding to a flow rate of 10-7 cm3/s. Blood viscosity in microvessels was assumed to be 2.2 cP (Pries, A. R., et al. (1994) Circ. Res. 75, 904-915). The computed estimates of wall shear stress are directly proportional to the assumed values of these two parameters, and the relative changes are unaffected by the assumed values.

Herein, a decrease in wall shear stress is indicative of focal microvessel dilatation formation. A decrease refers to a greater than a 10-fold decrease in wall shear stress as compared to the wall shear stress of normal microvessels, which ranges from 10 to 100 dyn/cm².

How to Monitor Lymphocyte Transmigration

Lymphocyte transmigration can be measured by any means known in the art, for example as referenced in, but not limited to, the following: West C A et al. (2001), Am. J. Physiol. Heart Circ. 281, H1742-H1750; West C A et al. (2001). Dev. Comp. Immunol. In press; West et al. (2001). J Immunol 166: 1517-1523; West C A, et al. (2001). Am. J. Physiol. Heart Circ. 281: H1742-H1750, West C A, et al. (2001). J. Histochem. Cytochem. 49: 511-518; West C A, et al. (2000). Transplantation Reviews 14: 225-236. At the focal region of a focal microvessel dilatation, lymphocytes transmigrate across the endothelium and form perivascular clusters. Herein, the presence of regional lymphocytic perivascular clusters is indicative of the presence of a focal microvessel dilatation.

In one embodiment lymphocytes are fluorescently labeled and tracked in vivo for periods much longer than their blood recirculation time of 3 to 5 hours. We have adapted recently developed thiol-reactive cytoplasmic dyes for use in our studies (West C A et al. (2001). J. Histochem. Cytochem. 49: 511-518.). These multi-colored dyes exist in the cytoplasm as fluorescent-peptide adducts so that they are retained in the cytoplasm for more than 72 hours at physiologic temperatures. Furthermore, these dyes are easily distinguishable by fluorescence microscopy, provide effective signal isolation for histologic analysis and are aldehyde fixable (West et al. (2001). J Immunol 166: 1517-1523; West C A et al. (2001). Am. J. Physiol. Heart Circ. 281: H1742-H1750; West C A, et al. (2001d). J. Histochem. Cytochem. 49: 511-518).

Second, studies using these cell tracers have demonstrated two significant features of lymphocyte recruitment. First, lymphocyte migration to the peripheral site of antigen-stimulation is independent of the lymph node of origin; that is, the frequency of lymphocytes migrating into the antigen-stimulated tissue is very similar whether the lymphocytes are from the stimulated lymph node or the contralateral control lymph node (West C A, et al. (2001) J Immunol 166: 1517-1523).

Studies in both the skin and lung demonstrated that lymphocyte recruitment into the tissue occurs in discrete clusters of cells. An explanation for this unexpected observation is that the injection of labeled lymphocytes functions as a “pulse” that enables us to visualize the migration pathway of lymphocytes in inflammation. In most conventional H&E histologic analyses, lymphocytes that have recently transmigrated are indistinguishable from those temporally removed from transmigration. It is speculated that lymphocytes migrating out of the tissue from these discrete areas subsequently percolate through the tissues and leave in the afferent lymph. Consistent with these observations, the longer the delay between injection of the lymphocytes and tissue harvest, the greater the distance from the microcirculation lymphocytes can be observed. These findings are consistent with focal areas of lymphocyte recruitment. Herein, lymphocyte clustering is consistent with focal areas of lymphocyte recruitment, and focal microvessel dilatation formation.

How to Monitor Endothelial Cell Proliferation.

Monitoring endothelial cell proliferation can also be used to assess the formation of focal microvessel dilatations.

Endothelial cell proliferation can be monitored by any means known in the art. In one embodiment, endothelial cell proliferation (and inhibition) will be assessed using serial immunohistochemistry of the inflammatory and control microcirculations using standard sereologic sampling techniques. Immunohistochemistry with the Ki-67 monoclonal antibody may be used to detect cell cycle progression. Counterstaining with CD31 or ICAM-2 monoclonal antibodies are used for endothelial localization controls. Intravital microscopy and microvascular corrosion casting with 3-dimensional scanning electron microscopy will provide a quantitative measure of the change in venular surface area.

In another embodiment, endothelial cell proliferation (and inhibition) will be assessed using a “checkerboard” assay. In this assay, an area of the skin is sheared and the biological mediators are applied (or injected) in a checkerboard pattern on the skin. At the conclusion of the experiment, “punch” biopsy samples of each mediator are obtained. The biopsies are simultaneously placed on a single slide for parallel immunohistochemical staining. The different conditions are internal controls for cell surface induction or inhibition. In the lung, a modified version of this assay will use segment-specific instillation of antigen to facilitate comparisons.

Screening Methods

Screening methods may be performed in vitro or in vivo. When assaying for inhibitors of focal microvessel dilatation formation, the structures are first induced using compounds known to promote formation of focal microvessel dilatations.

A. Induction of Focal Microvessel Dilatations

Conditions that “permit formation of a focal microvessel dilatation” are the natural physiological conditions present in a mammal. Focal microvessel dilatations can be induced in tissue using peptide-hapten antigens such as, but not limited to, oxazolone and TNP. Both alloantigens (and xenoantigens) and peptide-hapten antigens (e.g. oxazolone and TNBS/TNP) (West C A, et al. (2001). Dev. Comp. Immunol. In press; West C A, et al. (2001). J Immunol 166: 1517-1523) have successfully been used. The evidence to date suggests that the implications for focal microvessel dilatation development are the same for each of these antigens. More recent work has focused on the peptide-haptens oxazolone and TNP for several experimental reasons. These simple chemical compounds, often referred to as contact sensitizers or peptide-haptens, have several advantages for the study of the localized immune response. Foremost, peptide haptens demonstrate a unique capacity to trigger an intense cellular immune response. These molecules trigger a selective T lymphocyte infiltration in the tissue and paracortical hyperplasia in the draining lymph node (Hall J G (1980) Ciba Found Symp 71: 197-209.). Recent molecular studies have suggested that this unique “toxicity” is a result of a chemical modification of immunologically relevant proteins. The selective T-cell response may reflect hapten-modification of class I-restricted peptides (Handa K, and Herrmann S (1985) J Immunol 135: 1564-1572; Weltzien et al. (1992) Eur J Immunol 22: 863-866). More practical advantages include the ability to easily control the dose and route of antigen administration. In addition, the antigen can be applied by instilling the antigen into the airway or “painting” the skin: without surgery or injections that could result in unpredictable lymphatic drainage. Thus, peptide-haptens provide a potent trigger for T lymphocyte recruitment.

B. In Vitro and In Vivo Screening Methods

In order to contact the microcirculation with a candidate compound or agent, the compounds or agents can be administered by any means known in the art. Modes of administration include, but are not limited to, topically, intravenously, intraperitoneally, orally, intramuscularly, or subcutaneously.

In one embodiment, the screening methods are performed using the in vivo sheep model where the formation of focal microvessel dilatations has been first described. Upon compound administration, regional dilation of microvessels, lymphocyte velocity, wall shear stress, lymphocyte transmigration, and endothelial cell proliferation is monitored as described herein.

In another embodiment, an initial screen is performed in vitro by monitoring endothelial cell proliferation in an ex vivo skin section. Ex vivo skin that contains focal microvessel dilatations preferentially take up radionuclide markers of endothelial cell proliferation, such as ³H-thymidine (Bravermen et al. (1982) J Invest Dermatol Jan; 78 (1): 12-7). Herein, the skin section can be taken from any animal. When testing for inhibitors of focal microvessel dilatation formation, focal microvessel dilatations are first induced as described above. Potential inhibitory compounds are then administered to the excised skin section either by injection or by adsorption to the tissue to be examined. Compounds that potentially inhibit focal microvessel dilatation formation cause the tissue to have a reduced level of radionuclide incorporation as compared to control tissue that contains focal microvessel dilatations. Compounds that are identified in the initial screen can then be further tested in vivo in the sheep model described herein, or in any other appropriate animal.

In addition to the measurement of endothelial cell proliferation in excised tissue, such explants can also be used to monitor changes in the accumulation of perivascular lymphocytes in response to a test compound. For example, focal microvessel dilatations can be induced in the skin of an animal as described herein and the skin treated with or without a candidate modulator. An excised thick section of the tissue is then fixed and examined for differences in the accumulation or presence of perivascular lymphocytes, such differences indicating the activity of the candidate modulator. As used herein, a “thick section” or “whole mount” refers to a tissue section which can be as thick as the entire ear after the cartilage has been removed. The staining of vascular endothelium, either before or after harvesting the “whole mount” tissue section can reveal the focal dilatations of the blood vessels. Whole mount tissue sections can be used to correlate focal dilatation with evidence of vascular endothelial proliferation. In addition, evidence of perivascular lymphocytes can be detected in such sections, e.g., by microscopy, with or without staining for lymphocyte-specific markers known to those skilled in the art. Microvessel morphology in whole mount tissue sections can be examined using, e.g., lectin or other marker staining, as described by Thurston et al., 1999, Science 286: 2511-2514 for ear skin whole mounts. Briefly, after perfusion fixation, tissue is perfused with labeled lectin, e.g., biotinylated Lycopersicon esculentum lectin (Vector Laboratories, Burlingame, Calif., U.S.A.), which binds uniformly to the lumenal surface of endothelial cells and adherent leukocytes. The ears are removed and the skin separated from the cartilage. The ear skin whole mounts are permeabilized with 0.3% Trition X-100 and incubated in avidin-peroxidase complex overnight, before reacting with 0.5% ABC-3,3′-diaminobenzidine (DAB; Sigma, St. Louis, Mo., U.S.A.) and hydrogen peroxide. Stained ear skin is dehydrated through a series of alcohols, cleared in toluene, and mounted for microscopy with the dermal side up. Also, thinner sections of the excised thick section tissue explants can be prepared where necessary for microscopy.

In still another embodiment, an initial screen is performed in vitro or in vivo using an angiogenesis assay. Both focal microvessel dilatation formation and angiogenesis are linked with endothelial cell proliferation. Thus, the initial screening methods for drugs that can either inhibit or induce the formation of focal microvessel dilatations can be performed using known screening methods for identifying compounds that inhibit or induce angiogenesis. Herein, any in vitro or in vivo angiogenesis assay known in the art may be used. Compounds that are identified in the initial screen can then be further tested in vivo in the sheep model described herein, or in any other appropriate animal.

C. Angiogenesis Screening Assays

Examples of well described angiogenesis screening assays that may be used include, but are not limited to, in vitro endothelial cell assays, rat aortic ring angiogenesis assays, cornea micropocket assays, and chick embryo chorioallantoic membrane assays (Erwin, A. et al. (2001) Seminars in Oncology 28(6):570-576).

Some example in vitro endothelial cell assays include methods for monitoring endothelial cell proliferation, cell migration, or tube formation. Cell proliferation assays may use cell counting, BRdU incorporation, thymidine incorporation, or staining techniques (Montesano, R. (1992) Eur J Clin Invest 22: 504-515; Montesano, R. (1986) Proc Natl. Acad. Sci USA 83: 7297-7301; Holmgren L. et al. (1995) Nature Med 1: 149-153).

In the cell migration assays endothelial cells are plated on matrigel and migration monitored upon addition of a chemoattractant (Homgren, L. et al. (1995) Nature Med 1: 149-153; Albini, A. et al. (1987) Cancer Res. 47: 3239-3245; Hu, G. et al. (1994) Proc Natl Acad Sci USA 6: 12096-12100; Alessandri, G. et al. (1983) Cancer Res. 43: 1790-1797.)

The endothelial tube formation assays monitor vessel formation (Kohn, E C. et al. (1995) Proc Natl Acad Sci USA 92: 1307-1311; Schnaper, H W. et al. (1995) J Cell Physiol 165: 107-118).

Rat aortic ring assays have been used successfully for the screening of angiogenesis drugs (Zhu, W H. et al. (2000) Lab Invest 80: 545-555; Kruger, E A. et al. (2000) Invasion Metastas 18: 209-218; Kruger, E A. et al. (2000) Biochem Biophys Res Commun 268: 183-191; Bauer, K S. et al. (1998) Biochem Pharmacol 55: 1827-1834; Bauer, K S. et al. (2000) J Pharmacol Exp Ther 292: 31-37; Berger, A C. et al. (2000) Microvasc Res 60: 70-80.). Briefly, the assay is an ex vivo model of explant rat aortic ring cultures in a three dimensional matrix. One can visually observe either the presence or absence of microvessel outgrowths. The human saphenous angiogenesis assay, another ex vivo assay, may also be used (Kruger, E A. et al. (2000) Biochem Biophys Res Commun 268: 183-191).

Another common screening assay is the cornea micropocket assay (Gimbrone, M A. et al. (1974) J Natl Canc Inst. 52: 413-427; Kenyon, B M. et al. (1996) Invest Opthalmol Vis Sci 37: 1625-1632; Kenyon, B M. et al. (1997) Exp Eye Res 64: 971-978; Proia, A D. et al. (1993) Exp Eye Res 57: 693-698). Briefly, neovascularization into an a vascular space is monitored in vivo. This assay is commonly performed in rabbit, rat, or mouse.

The chick embryo chorioallantoic membrane assay has been used often to study tumor angiogenesis, angiogenic factors, and antiangiogenic compounds (Knighton, D. et al. (1977) Br J Cancer 35: 347-356; Auerbach, R. et al. (1974) Dev Biol 41: 391-394; Ausprunk, D H. et al. (1974) Dev Biol 38: 237-248; Nguyen, M. et al. (1994) Microvasc Res 47: 31-40). This assay uses fertilized eggs and monitors the formation of primitive blood vessels that form in the allantois, an extra-embryonic membrane.

The above is just a sampling of angiogenic factor and angiogenic inhibitor assays that may be used as an initial screen for agents or compounds that inhibit or induce focal microvessel dilatation formation.

Treatment

A. Diseases

The present invention will identify compounds or agents that inhibit focal microvessel dilatation formation that can be used in the treatment of a variety of lymphocytic inflammatory disorders. For example autoimmune diseases, such as, articular rheumatism, systemic lupus erythematosus, Sjoegren syndrome, multiple sclerosis, myasthenia gravis, type I diabetes mellitus, endocrine ophthalmic disease, primary biliary cirrhosis, Crohns disease, glomerular nephritis, sarcoidosis, psoriasis, eczema, atopic dermatitis, pityriasis rosea, mycosis, fungoides, lichen planus, and granuloma annulare, variola, hypoplastic anemia, idiopathic thrombocytopenic purpura, rheumatoid arthritis, and the like. Compounds or agents that inhibit focal microvessel dilatation formation further provide for treatment and prevention of graft vs host disease, as well as, viral diseases, such as Herpes Simplex Virus, Varicella, and Herpes Zoster. Inhibitors of focal microvessel dilatation formation inhibit lymphocytic infiltration to the site of inflammation.

The present invention further provides for a method for treatment of lymphocytic inflammation using a combination therapy of anti-angiogenic compounds and inhibitors of lymphocyte cell-cell adhesion. Herein, it is shown that anti-angiogenic compounds inhibit focal microvessel dilatation formation thereby inhibiting lymphocyte transmigration across the vascular endothelium. Combination therapy using compounds or agents that have two different mechanisms of action can substantially increase the potency of any given therapy. In the present invention, anti-angiogenic compounds are combined with inhibitors of lymphocyte cell-cell adhesion in order to inhibit lymphocyte transmigration.

In addition, herein it is disclosed that pro-angiogenic factors that induce endothelial cell proliferation can be used to potentiate focal microvessel dilatation formation thereby increasing lymphocyte transmigration. The pro-angiogenic factors may be used to induce formation of focal microvessel dilatations in order to promote lymphocyte infiltration of a tumor and subsequent tumor cell death.

Compounds or factors that induce formation of focal microvessel dilatations, of the present invention such as pro-angiogenic factors, may be used to treat a variety of cancers. Examples of treatable cancers include, but are not limited to, skin cancer, head and neck tumors, breast tumors, colon or bladder cancer, and Kaposi's sarcoma.

B. Dosage Formulation and Administration

When the amount of a compound or other agent to be administered to an animal is considered, it will be apparent to those of skill in the art that the effective amount of a composition administered in the invention will depend, inter alia, upon the efficiency of cellular uptake of a composition, the administration schedule, the unit dose administered, whether the compositions are administered in combination with other agents, the health of the recipient, and the biological activity of the particular composition.

The pro-angiogenic factors, anti-angiogenic factors, and other compounds or agents of the present invention may be administered either alone, or in combination with a pharmaceutically or physiologically acceptable carrier, excipients or diluents. Generally, such carriers should be non-toxic to recipients at the dosages and concentrations employed. The anti-, pro-angiogenic factors, or pharmaceutical compositions provided herein may be prepared for administration by a variety of different routes, including for example intrarticularly, intraocularly, intranasally, intradermally, sublingually, orally, topically, intravesically, intrathecally, topically, intravenously, intraperitoneally, intracranially, intramuscularly, subcutaneously, or even directly into a tumor or disease site. The route of administration and the dosage regimen will be determined by skilled clinicians, based on factors such as the exact nature of the condition being treated, the severity of the condition, the age and general physical condition of the patient and so on.

The dosages for treatment of the above-mentioned disorders will be dependent on the route of administration. The dosage may be ascertained through the use of established assays and determining dosages using appropriate dose response data. Skilled clinicians will determine whether the compounds or agents administered are effective in treatment of the above-mentioned diseases, and can adjust the dosages accordingly. Effective amounts of the compositions of the invention can vary from 0.01-1,000 mg per kg of body weight, although lesser or greater amounts can be used.

### EXAMPLES

The invention will now be further illustrated with reference to the following examples. It will be appreciated that what follows is by way of example only and that modifications to detail may be made while still falling within the scope of the invention.

Observing lymphocyte Migration through the Inflammatory Microcirculation

Lymphocyte migration through the inflammatory microcirculation is directly observed with a custom-designed epi-illumination system that has a super high pressure mercury lamphouse for the delivery of light through the optical system as bright-field, dark-field, or fluorescence illumination. The fluorescent filter block used in these experiments was an orange (DM 560 nm) filter. The Nikon epi-achromat objectives were 10× and 20× magnification. The intravital microscopy was performed using a custom machined titanium stage (MicroSurg, Boston Mass.) that directly attached to the microscope stand to limit vibration. The tissue surface of the window was composed of a 0.635″ flange surrounding the window lens. Within the flange, two concentric 2.5 mm vacuum galleries provided tissue apposition to the lens surface without compression of the tissue and with minimal circulatory disturbances. The window lens (1.275″×0.059″) was designed with ultra-violet grade fused silica (Kreischer Optics; McHenry, Ill.). The surfaces were optically polished to retical quality (20-10 scratch-dig per MIL-O-13830A). The camera was a Dage-MTI CCD-72 series high resolution CCD imager and high performance analog processor with 768×493 active elements and 570 TVL resolution (Dage-MTI, Michigan City Ind.). The image was intensified using a GenIIsys optically coupled image intensifier (Dage-MTI, Michigan City Ind.).

Intravital videomicroscopy images were recorded on a Panasonic model AG-6750A S-VHS video recorder (Secaucus, N.J.)(30 frame/second) with horizontal resolution of 400 lines. Time base correction was performed using a TBC III board (VT2500, Digital Processing Systems, Florence, Ky.). Video of the recorded images was processed through a M-Vision 1000 PCI bus frame grabber (Mutech, Waltham Mass.) in a Pentium III (700 megaherz, 256 megabyte RAM) computer running the MetaMorph Imaging System 4.0 (Universal Imaging, Brandywine, Pa.) under Microsoft Windows NT (Redmond, Wash.). Image stacks were routinely created from 12 second to 5 minute video sequences. The image stacks were processed with standard MetaMorph filters. After routine distance calibration and thresholding, the “stacked” image sequence was measured using the MetaMorph's object tracking and integrated morphometry applications.

In the experimental method, the epicutaneous antigen oxazolone was used in the sheep model to stimulate lymphocyte recruitment out of the skin microcirculation (West et al., J Immunol 166, 1517-23. (2001)). Previous work in this model has shown that the peak of lymphocyte recruitment occurs 96 hours after the application of oxazolone. Ox. Randomly bred sheep, ranging in weight from 25 to 35 kg, were used in these studies. Sheep were excluded from the analysis if there was any gross or microscopic evidence of dermatitis. The sheep were given free access to food and water. The care of the animals was consistent with guidelines of the American Association for Accreditation of Laboratory Animal Care (Bethesda Md.). The sheep ear and neck region was sheared bilaterally and the lanolin removed with an equal mixture of ether (J T Baker, Phillipsburg N.J.) and ethanol (AAPER, Shelbyville Ky.). The antigen, a 5% solution of 2-phenyl-4-ethoxymethylene-5-oxazolone (oxazolone) (Sigma, St. Louis Mo.), was sprayed onto the ear and a localized region of the neck as a 4:1 oxazalone:olive oil mixture using a syringe and 23 gauge needle. A vehicle only control was applied to the contralateral skin.

Regional efferent lymphocytes were fluorescently labeled and re-injected into the inflammatory microcirculation. The precapsular lymph node, with a lymphatic drainage basin including the ear and neck, was used for all efferent lymph duct cannulations. The efferent lymph duct was cannulated with a heparin-bonded polyurethane catheter (Solo-Cath, CBAS-C35; Setters Life Sciences, San Antonio Tex.). The cannula was passed through a 5 cm subcutaneous tunnel and secured at the skin. The lymph was collected in 50 cc sterile centrifuge tubes (Falcon, Franklin Lakes N.J.) containing 200 IU of heparin, 2000 IU of penicillin (Cellgro, Mediatech, Inc.; Herndon Va.), and 2000 ug of streptomycin (Cellgro). The lymph cells were labeled with succinimidyl esters of the mixed isomer preparation of 5-(and-6)-carboxytetrmethylrhodamine (5(6)-TAMRA)(ex 540 nm/em 565 nm; Molecular Probes, Eugene Oreg.). Prior to labeling, the lymph cells were washed three times in Dulbecco's Modified Eagle's Medium (DME) with 2,000 mg/L glucose (Sigma, St. Louis, Mo.) and resuspended in phosphate buffered saline (PBS) containing 25 ul of the stock 5(6)-TAMRA fluorescent dye. The cells were incubated for 15 minutes at room temperature and washed in cold DMEM. The cells were resuspended in room temperature PBS at 0.7-5.0×10⁷ cells/ml prior to injection into the common carotid arteries proximal to the origin of the external auricular arteries. The common carotid arteries were exposed and cannulated with a heparin-bonded polyurethane catheter (Solo-cath, CBAS-C35, Setters Life Sciences, San Antonio Tex.). The catheter was tunneled through the subcutaneous tissue to the dorsum of the neck and secured. The catheter was fitted with a stub-nose adapter and flushed with heparinized saline (100 units/ml)(Elkins-Sinn, Cherry Hill N.J.).

These migratory cells were tracked through the inflammatory microcirculation using epi-fluorescence intravital videomicroscopy.

### Example 1

### Lymphocyte Slowing and Transmigration in Focal Microvessel Dilatations

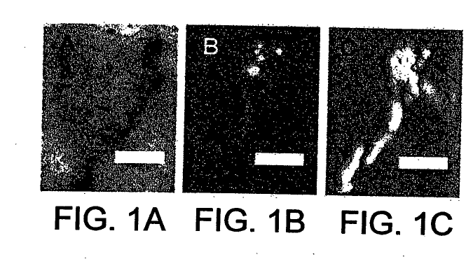

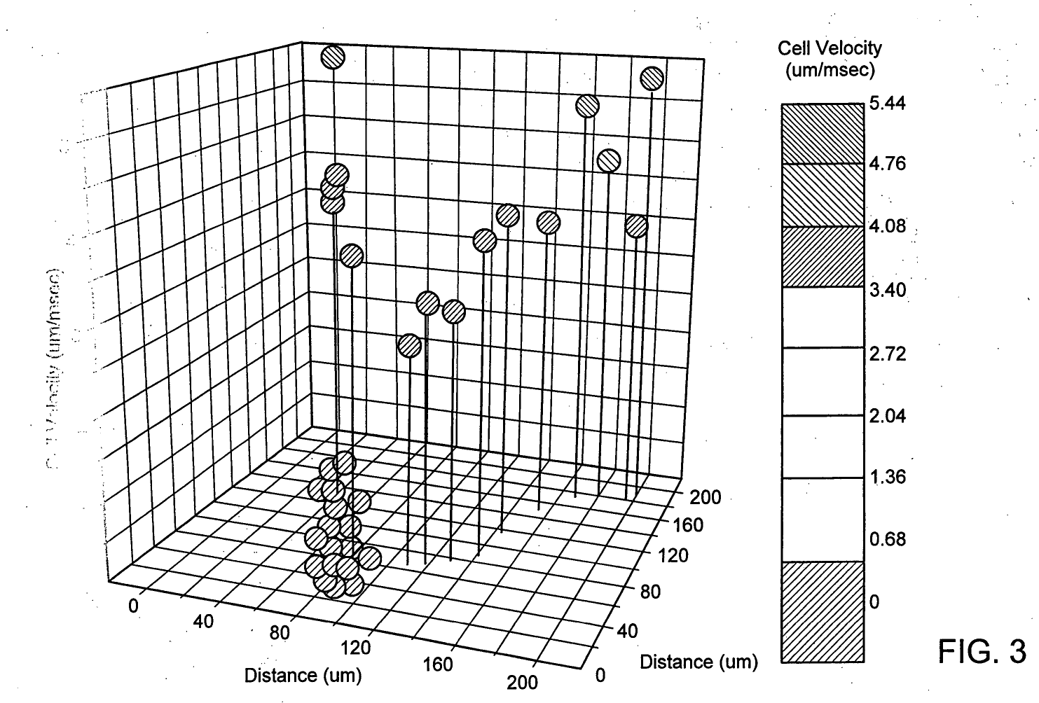

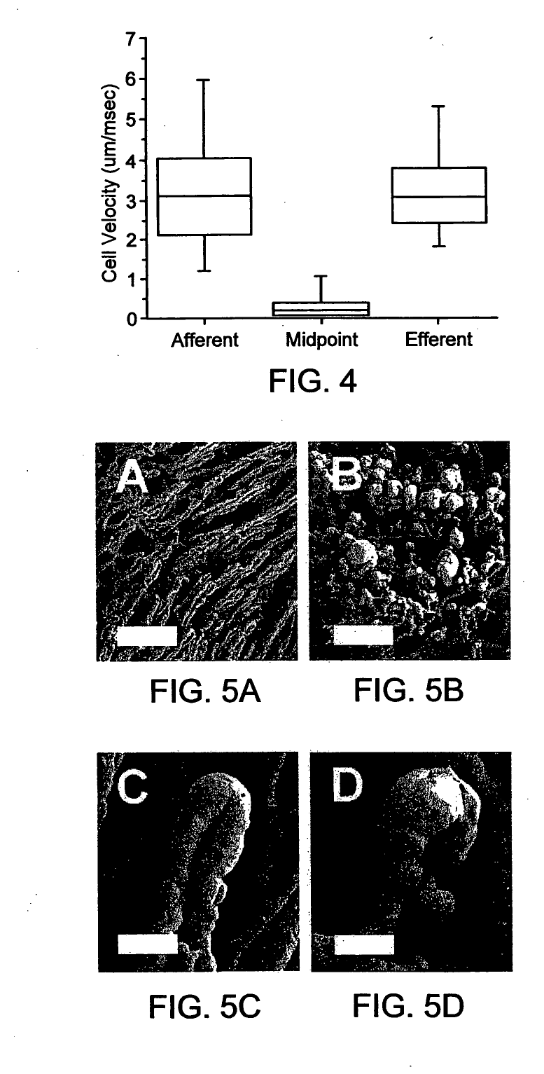

The intravital videomicroscopy studies demonstrated reproducible lymphocyte slowing in focal regions of the microcirculation (FIG. 1). Lymphocytes in these regions demonstrated a greater than 10-fold reduction in flow velocity (FIGS. 2 and 3). After the cells passed through these vascular segments, they rapidly returned to baseline flow velocities (FIG. 4). Also suggesting discrete structural changes in the sheep skin microcirculation, the regions of lymphocyte slowing were identified at approximately 100 um intervals (FIG. 1). The focal areas defined not only areas of lymphocyte slowing, but also the regions of lymphocyte transmigration. The focal areas of lymphocyte slowing were the only regions of the superficial vascular plexus where lymphocyte transmigration was observed (FIG. 1) (West et al. (2001), Am. J. Physiol. Heart Circ. 281, H1742-H1750. These findings suggested that lymphocyte transmigration involved structural adaptations in the inflammatory microcirculation.

### Example 2

### Focal Microvessel Dilatation Morphology

To evaluate the morphology of these focal regions of lymphocyte slowing and transmigration, corrosion cast injections of the inflammatory microcirculation were performed. The corrosion casts were examined by scanning and transmission electron microscopy and evaluated by digital morphometry. After systemic heparinization with 750 u/kg intravenous heparin, the external auricular arteries were bilaterally cannulated and perfused with approximately 100 cc of 37° C. saline followed by a 2.5 percent buffered glutaraldehyde solution (Sigma) at pH 7.40. The casts were made by perfusion of the ear arteries with 100 cc of Mercox (SPI, West Chester Pa.) diluted with 20 percent methylmethacrylate monomers (Aldrich Chemical, Milwaukee Wis.). After complete polymerization, the ears were harvested and macerated in 5% potassium hydroxide followed by drying and mounting for scanning electron microscopy. The microvascular corrosion casts were imaged after coating with gold in Argon atmosphere with a Philips ESEM XL30 scanning electron microscope. Stereo-pair images were obtained by using tilt angles from 6° to 20°. Diameters were interactively measured orthogonal to the vessel axis after storage of calibrated images, using ANALYSIS software (version 2.1). The quality of the corrosion casts was controlled by examining semithin light microscopic sections stained with methylene blue. The corrosion casts demonstrated filling of the whole capillary bed from artery to vein without evidence of extravasation or pressure distension. Judged on the basis of previous work (Su, et al., 2001, Transplantation 72, 516-522), shrinkage of the corrosion casts was on the order of 6%.

Scanning electron microscopy of the inflammatory microcirculation 96 hours after oxazolone stimulation showed focal dilatation in the superficial vascular plexus (FIG. 5). The focally dilated vascular segments, referred to as focal microvessel dilatations or microangiectasias, ranged up to 90 um in diameter and were located at approximately 100 um intervals, corresponding to the observed spacing of the regions of lymphocyte slowing. In contrast, the dilated segments were rare in the control microcirculation. Vascular diameters averaged 11.3±3.3 μm (mean±SD, n=58) in the afferent segment, 27.4±9.6 μm in the dilated segment, and 16.3±5.4 μm in the efferent segment. In control tissues, corresponding diameters were 11.1±3.1 μm in the afferent segment (n=58), 15.3±4.0 μm in the tip of the loop and 11.7±2.9 μm in the efferent segment. Diameter increases in the tip of the loop and efferent segment of inflammatory tissue relative to control were significant (p<0.001: Mann-Whitney Rank Sum Test). The dilated microvessels were morphologically most consistent with capillary sinusoids and appeared to be present at the transition point between the capillary and postcapillary venule.

### Example 3

### Focal Microvessel Dilatation Wall Shear Stress and Microhemodynamic Mapping of Focal Microvessel Dilatations

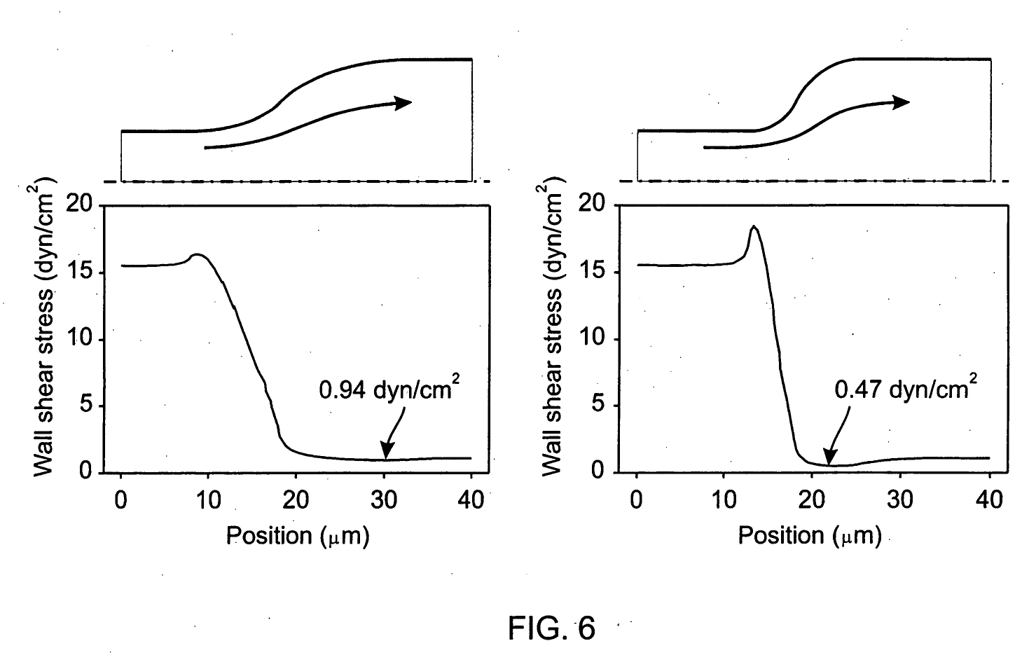

The focal structural changes and the reduction in flow velocity indicated that focal microvessel dilatations have a significant impact on wall shear stress in the microcirculation. To define the microhemodynamic implications of the focal microvessel dilatations, the corrosion casts of the inflammatory skin were evaluated by quantitative 3-dimensional (3-D) scanning electron microscopy (Konerding, et al. (2001), Br J Cancer 84, 1354-62, M. A. Konerding et al. (1999), Br J Cancer 80, 724-32). Based on these data, 3-D hemodynamic maps of the focal microvessel dilatations were calculated. Wall shear stresses in the focal microvessel dilatations demonstrated a greater than 10-fold reduction in wall shear stress (FIG. 6). To estimate wall shear stresses, finite-element computations of flow fields in the neighborhood of the transition from the afferent vessel to the dilated segment were performed (FIG. 6). Axisymmetric geometries were assumed, with diameters corresponding to the mean measured values. Two different transition profiles, gradual and abrupt, were considered, representative of the range of shapes seen in the SEMs. Computed wall shear stresses declined by a factor of more than 10 from the afferent segment to the focal microvessel dilatation. Lowest levels occurred immediately inside the entrance to the dilated region and were below 1 dyn/cm2. For a given flow rate, a more abrupt transition in diameter resulted in a lower minimum shear stress. Corresponding calculations for control tissues predicted wall shear stresses above 5 dyn/cm² in the tip of the loop.

### Example 4

### Anti-Angiogenic Factor Inhibits Lymphocyte Transmigration

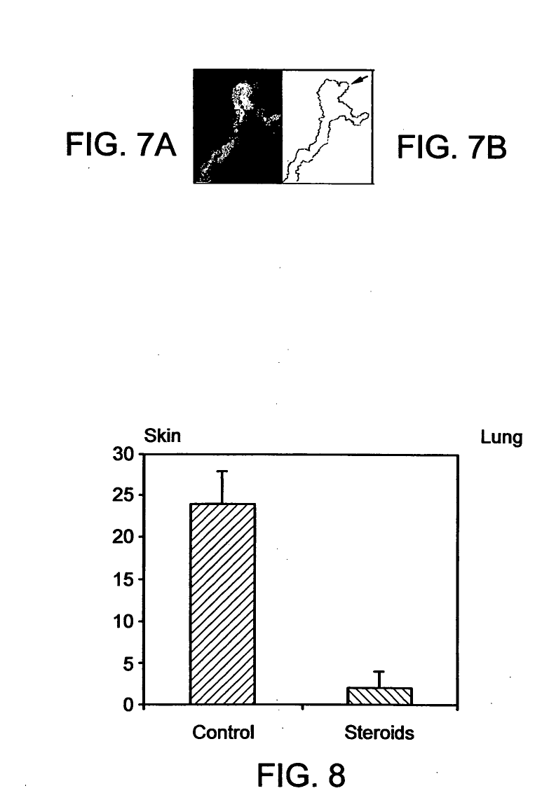

Lymphocyte slowing and transmigration occurs in focal regions of the microcirculation defined by dilated microvessels. Further, the dilated areas are associated with endothelial cell proliferation. These data suggested that the inhibition of endothelial cell proliferation should decrease focal microvessel dilatation prevalence and lymphocyte recruitment. To date, only topical steroids have been used. Upon administration of steroid to sheep skin, a marked reduction in the number of lymphocytes migrating into the antigen-stimulated tissue was observed (FIG. 8). FIG. 8 shows the number of recruited cells per 200×400 um grid in the inflammatory and steroid-treated skin.

The foregoing examples demonstrate experiments performed and contemplated by the present inventors in making and carrying out the invention. It is believed that these examples include a disclosure of techniques which serve to both apprise the art of the practice of the invention and to demonstrate its usefulness. It will be appreciated by those of skill in the art that the techniques and embodiments disclosed herein are preferred embodiments only that in general numerous equivalent methods and techniques may be employed to achieve the same result.

All of the references identified herein above are hereby expressly incorporated herein by reference to the extent that they describe, set forth, provide a basis for or enable compositions and/or methods which may be important to the practice of one or more embodiments of the present inventions. All applications, patents and literature references cited in the specification are hereby incorporated by reference, in their entireties including figures and tables.

1. A screening method for an agent or compound that inhibits the formation of focal dilatations in microvessels, comprising:
contacting the microcirculation with a candidate compound under conditions that permit the formation of focal dilatations; and
detecting whether a focal dilatation is formed, wherein the failure to form a focal dilatation is indicative of the inhibitory activity of said candidate compound.

2. The method of claim 1, the method further comprising the step of administering said compound to an animal and determining whether focal microvessel dilatation formation is inhibited at a site of inflammation.

3. The method of claim 1, wherein said contacting comprises applying said compound to the skin.

4. The method of claim 3, further comprising the step, after the step of contacting the microcirculation with a candidate compound under conditions that permit the formation of focal dilatations, of removing a thick section of said skin and performing said detection on said thick section.

5. The method of claim 2 wherein inhibition of focal microvessel dilatation reduces the accumulation of perivascular lymphocytes at said site of inflammation.

6. The method of claim 2, further comprising administering an inhibitor of lymphocyte cell-cell adhesion.

7. A screening method for an agent or compound that inhibits the formation of focal dilatations in microvessels, comprising:
contacting the microcirculation with a candidate compound under conditions that permit the formation of focal dilatations in microvessels; and
detecting one or more of the following:
a) substantially no focal reduction in blood cell flow velocity within the microvasculature;
b) substantially no reduction in wall shear stress within the microvasculature;
c) substantially no focal increase in the diameter of the microvasculature;
d) substantially no increase in extravascular lymphocytes; and
e) substantially no increase in endothelial cell proliferation,
wherein the detection of at least one of (a), (b), (c), (d) or (e), relative to microcirculation not contacted with said candidate compound, indicates that said candidate compound inhibits the formation of focal dilatations in microvessels.

8. The method of claim 7 wherein said flow velocity is detected for a blood cell which is one or more of a lymphocyte, neutrophil or red blood cell.

9. The method of claim 8 wherein said lymphocyte, neutrophil or red blood cell is detectably labeled.

10. The method of claim 9 wherein said lymphocyte, neutrophil or red blood cell is fluorescently labeled.

11. The method of claim 7 further comprising the step, before said detecting step, of removing a thick section of tissue containing the microcirculation contacted with said compound, wherein said detecting is performed on said thick section.

12. A screening method for an agent or compound that induces the formation of focal dilatations in microvessels, comprising:
contacting the microcirculation with a candidate compound under conditions that permit the formation of focal dilatations in microvessels; and
detecting whether a focal dilatation in a microvessel is formed, wherein the formation of a focal dilatation in a microvessel is indicative of the induction activity of said compound.

13. The method of claim 12, said method further comprising the step of administering said compound to an animal with a tumor and detecting the formation of focal microvessel dilatations adjacent to said tumor.

14. The method of claim 13 wherein the induction of said focal microvessel dilatations increases perivascular lymphocyte accumulation within said tumor.

15. A screening method for an agent or compound that induces the formation of focal dilatations in microvessels, comprising:
contacting the microcirculation with a candidate compound under conditions that permit the formation of focal dilatations in microvessels; and
detecting one or more of the following:
a) a reduction in blood cell flow velocity within the microvasculature;
b) a reduction in wall shear stress within the microvasculature;
c) a focal increase in the diameter of the microvasculature;
d) an increase in extravascular lymphocytes; and
e) an increase in endothelial cell proliferation,
wherein the detection of one or more of (a), (b), (c), (d) or (e), relative to microcirculation not contacted with said candidate compound, indicates that said candidate compound induces the formation of focal dilatations in microvessels.

16. The method of claim 15 wherein said flow velocity is detected for a blood cell which is one or more of a lymphocyte, neutrophil or red blood cell.

17. The method of claim 16 wherein said lymphocyte, neutrophil or red blood cell is detectably labeled.

18. The method of claim 17 wherein said lymphocyte, neutrophil or red blood cell is fluorescently labeled.

19. The method of claim 15 further comprising the step, before said detecting step, of removing a thick section of tissue containing the microcirculation contacted with said compound, wherein said detecting is performed on said thick section.

20. A method of screening for a compound that modulates the accumulation of extravascular lymphocytes, the method comprising:
a) contacting a tissue with an agent that induces inflammation,
b) contacting said tissue with a candidate modulator compound, and
c) detecting a difference in one or more of the following:
i) local accumulation of extravascular lymphocytes in said tissue;
ii) the size or number of focal microvessel dilatations in microvessels in said tissue;
iii) localized blood cell flow velocity in a microvessel of said tissue;
iv) wall shear stress within the microvasculature; and
v) endothelial cell proliferation;
relative to the detection of one or more of (i), (ii), (iii), (iv) and (v) occurring in the absence of said candidate compound, wherein said difference indicates that said candidate compound modulates the accumulation of extravascular lymphocytes.

21. The method of claim 20 wherein said difference comprises a decrease in one or more of (i), (ii) and (v), and/or an increase in one or both of (iii) and (iv), and wherein said difference indicates that said candidate modulator is an inhibitor of the accumulation of extracellular lymphocytes.

22. The method of claim 20 wherein said difference comprises an increase in one or more of (i), (ii) and (v), and/or a decrease in one or both of (iii) and (iv), and wherein said difference indicates that said candidate modulator is an inducer of the accumulation of extracellular lymphocytes.

23. The method of claim 20 wherein said difference in blood cell flow velocity is detected for a blood cell which is one or more of a lymphocyte, neutrophil or red blood cell.

24. The method of claim 20 wherein said difference in blood cell flow velocity is measured using intravital video microscopy.

25. The method of claim 23 wherein said lymphocyte, neutrophil or red blood cell is detectably labeled.

26. The method of claim 25 wherein said lymphocyte, neutrophil or red blood cell is fluorescently labeled.

27. A method of validating a candidate compound as an anti-inflammatory compound, the method comprising:
a) contacting a tissue with an agent that induces inflammation;
b) contacting said tissue with a compound identified as a candidate anti-inflammatory compound in a high throughput assay;
c) detecting a difference in local accumulation of extravascular lymphocytes in said tissue relative to the local extravascular lymphocytic accumulation occurring in the absence of said candidate compound, wherein said difference validates said candidate compound as an anti-inflammatory compound.

28. The method of claim 27 wherein said high throughput assay comprises microarray hybridization.

29. A method for treating a tumor in a patient by administering a pro-angiogenic factor in an amount sufficient to induce the formation of focal microvessel dilatations and lymphocytic infiltration of said tumor.

30. A method of treating a patient suffering from a disease involving inflammation comprising administering an inhibitor of angiogenesis and an inhibitor of lymphocyte cell-cell adhesion in an amount sufficient to inhibit the formation of focal microvessel dilatations and lymphocytic infiltration into the site of inflammation.

31. A method for immunosuppression in a patient comprising administering an angiogenesis inhibitor and an inhibitor of lymphocyte cell-cell adhesion in an amount sufficient to inhibit the formation of focal microvessel dilatations and lymphocytic infiltration of a site of inflammation.

32. The method of claim 30 wherein said inhibitor of lymphocyte cell-cell adhesion is selected from the group consisting of: an inhibitor of one of LFA-1, I-CAM 1, or L-selectin.

33. The method of claim 31 wherein said inhibitor of lymphocyte cell-cell adhesion is selected from the group consisting of: of an inhibitor of one of LFA-1, I-CAM 1, or L-selectin.

34. The method of claim 31 wherein said patient has a transplanted organ.

35. The method of claim 30 wherein the patient suffers from a disease selected from the group consisting of: psoriasis, eczema, atopic dermatitis, pityriasis rosea, mycosis, fungoides, lichen planus, and granuloma annulare.

36. A method of claim 30 wherein the patient has an autoimmune disease.
